(19) United States
(12) Patent Application Publication
(10) Pub. No.: US 2012/0165659 A1
(43) Pub. Date: Jun. 28, 2012

(54) RADIOPAQUE IMPLANT

(75) Inventors: Jianmin Li, Lexington, MA (US); Hamid Davoudi, Westwood, MA (US); Timothy P. Harrah, Cambridge, MA (US); Brent Palmisano, Fiskdale, MA (US)

(73) Assignee: BOSTON SCIENTIFIC SCIMED, INC., Maple Grove, MN (US)

(21) Appl. No.: 13/312,750

(22) Filed: Dec. 6, 2011

Related U.S. Application Data
(60) Provisional application No. 61/426,355, filed on Dec. 22, 2010.

(51) Int. Cl. A61B 5/05; D03D 11/00; D03D 1/00; A61B 17/03

(52) U.S. Cl. 600/424; 606/151; 139/410; 139/420 R

(57) ABSTRACT

In some embodiments, an implant includes a substrate and a radiopaque portion. The radiopaque portion is coupled to the substrate. The radiopaque portion has a first portion extending along a first axis and a second portion extending along a second axis different than the first axis. In some embodiments, the substrate has a perimeter that defines a shape having a first size. The radiopaque portion forms a shape having a second size. The shaped formed by the radiopaque portion is substantially the same as the shape defined by the perimeter of the substrate. The first size is greater than the second size.

### CROSS REFERENCE TO RELATED APPLICATION

This application is a Nonprovisional of, and claims priority to, U.S. Provisional Application No. 61/426,355, filed on Dec. 22, 2010, entitled “RADIOPAQUE IMPLANT”, which is incorporated by reference herein in its entirety.

### TECHNICAL FIELD

This disclosure relates generally to bodily implants and more particularly to bodily implants that include features for determining an orientation of an implant while the implant is disposed within the body of the patient.

### BACKGROUND

A variety of medical procedures include placing implants within a body of a patient. Some medical procedures include placing implants within a body of a patient such that the implant provides support to a portion of the body of the patient. For example, some medical procedures are performed to treat various female pelvic dysfunctions, including procedures to treat urinary incontinence, and correcting various prolapse conditions such as uterine prolapse, cystoceles, rectoceles, and vaginal vault prolapse.

Some of the implants that are placed within a body of a patient are formed of a plastic material. For example, some implants are formed of a plastic mesh material.

Often times it is desirable to view an implant that has been disposed within a body of a patient. For example, it may be desirable to view an implant during an implantation procedure to confirm that the implant is located in the correct location within the body. Additionally, it may be desirable to view an implant after an implantation procedure to confirm that the implant has not moved from its implanted position, has not shrunk, or has not otherwise changed its orientation within the body of the patient.

Implants disposed within a body of a patient can be viewed using imaging devices such as x-ray devices. Some known implants, however, may not allow for the monitoring and determining of the status and orientation of the implant within the body of the patient.

Accordingly, it would be desirable to provide an implant that could be viewed with an imaging device after an implantation procedure to determine the status and/or orientation of the implant within the body of the patient.

### SUMMARY

In some embodiments, an implant includes a substrate and a radiopaque portion. The radiopaque portion is coupled to the substrate or is a portion of the substrate. The radiopaque portion has a first portion extending along a first axis and a second portion extending along a second axis different than the first axis. In some embodiments, the substrate has a perimeter that defines a shape having a first size. The radiopaque portion forms a shape having a second size. The shape formed by the radiopaque portion is substantially the same as the shape defined by the perimeter of the substrate. The first size is greater than the second size.

### BRIEF DESCRIPTION OF THE DRAWINGS

FIG. 1 is a schematic illustration of an implant according to an embodiment.

FIGS. 2-5 are top views of implants according to different embodiments.

FIG. 6 is a flow chart of a method of forming an implant according to an embodiment.

FIG. 7 is a flow chart of a method of detecting an implant according to an embodiment.

### DETAILED DESCRIPTION

The devices and methods described herein are generally directed to implants and medical devices that are configured to be placed within a body of a patient. In some embodiments, the implants are placed with a body of a patient and positioned to provide support to a portion of the body of the patient. For example, in some embodiments, the implants include, but are not limited to, implants that are placed within a pelvic region of a patient. Such implants can be placed into the pelvic space of a patient and secured at any of several locations within the pelvic space to treat many different pelvic floor dysfunctions. For example, an implant can be secured to a sacrospinous ligament or a ureterosacral ligament for uterine preservation (e.g., if a prolapsed uterus is otherwise healthy, a hysterectomy is not preformed and the uterus is re-suspended with an implant), or for posterior support. In another embodiment, an implant can be secured to pubo-urethral tissue or an obturator muscle (e.g., internus or externus) or membrane (each also referred to herein as “obturator”) to treat, for example, incontinence. In yet another embodiment, an implant can be secured to a sacrospinous ligament or an arcus tendineus fascia pelvis (i.e., white line) (also referred to herein as “arcus tendineus”) for paravaginal repairs including, for example, cystoceles, rectoceles and enteroceles. An implant can also be secured to various combinations of such locations.

While many of the implants described in detail herein may be placed in a pelvic region of a patient, the invention may be used in any type of bodily implant (i.e., implants that may be disposed or placed in any location within the body of the patient). Additionally, the invention may be incorporated in implants that may be placed or disposed within a female patient or a male patient.

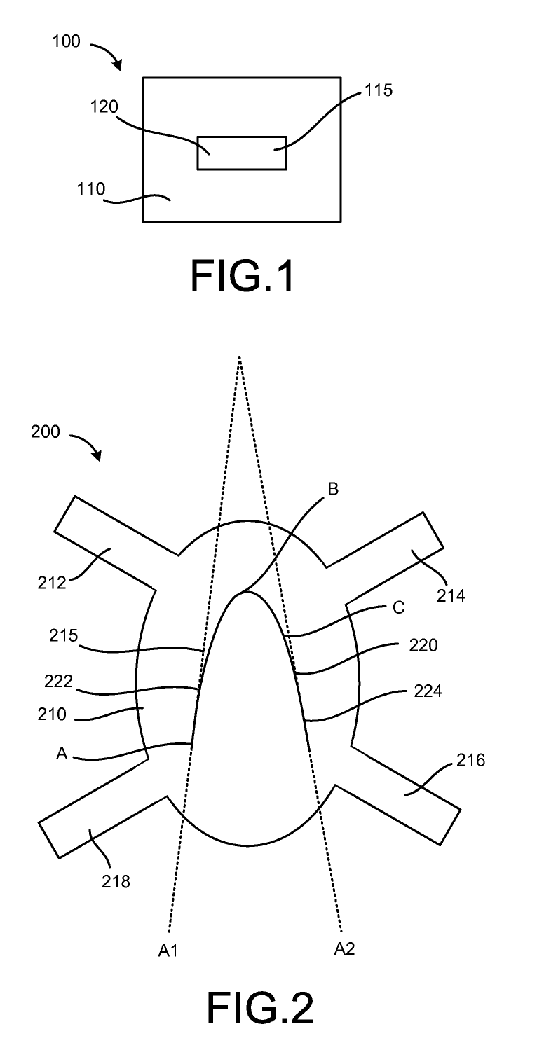

FIG. 1 is a schematic illustration of an implant 100 according to an embodiment of the invention. The implant includes a substrate 110 and a radiopaque portion 115. The radiopaque portion 115 is configured to be visible with an imaging device. In some embodiments, the radiopaque portion 115 includes a radiopaque member 120. In some embodiments, the implant 100 is configured to be placed with a body of a patient. For example, in some embodiments, the implant 100 is configured to be placed within a body of a patient and configured to provide support to a portion of the body of the patient. In other embodiments, the implant 100 is configured to be placed within a body of a patient for a purpose other than providing support to a portion of the body.

For example, in some embodiments, the implant 100 may include arm members configured to extend to or through bodily tissue to help secure the implant within the body of the patient. For example, in some embodiments, the substrate 110 may include arm members or portions that include tangs to help secure the implant 100 in place within the body of the patient. The terms “tanged” or “tangs” as used herein mean roughened or jagged edges or areas, such as can result from cutting a woven or knit mesh material. In other embodiments, the substrate 110 may include barbs, dimples and/or other protrusions configured to engage the bodily tissue of the patient to help retain the implant 100 in place within the body of the patient. In other embodiments, other mechanisms may be used to couple the implant 100 to bodily tissue. For example, sutures may be used to couple the implant 100 to bodily tissue to help support the implant 100 within the body of the patient.

The substrate 110 can be monolithically formed or alternatively, the substrate 110 can be formed with multiple different materials and/or can include multiple different components or portions coupled together. In some embodiments, the substrate 110 can be formed with a combination of materials including synthetic and biological materials. In other embodiments, the substrate 110 is formed with a biological material.

The substrate 110 may be of any size or shape. For example, in some embodiments, the substrate 110 is sized and shaped to be inserted within a body of a patient and to provide support to a portion of the body of the patient. For example, in some embodiments, the substrate 110 is sized and shaped to be inserted into a pelvic region of a patient. In other embodiments, the substrate 110 is sized and shaped to be inserted into other portions of the body of the patient.

The substrate 110 may be formed of any type of biocompatible material, such as a biocompatible synthetic material or a natural, biologic material. In some embodiments, the substrate 110 is formed of a biocompatible synthetic material.

For example, in some embodiments, the substrate 110 is formed of or includes a synthetic mesh material. For example, in some embodiments, the substrate 110 is a formed of a polypropylene mesh material. In some embodiments, some or all of an substrate 110 can be formed with the Advantage™ Mesh or the Polyform™ Synthetic Mesh material each provided by Boston Scientific Corporation. In other embodiments, the substrate 110 is a mesh of another biocompatible synthetic material.

In the illustrated embodiment, the radiopaque portion 115 includes a radiopaque member 120. The radiopaque member 120 is coupled to the substrate 110. In some embodiments, the radiopaque member 120 is a filament such as a radiopaque suture or a radiopaque thread. For example, in some embodiments, the radiopaque member 120 is a suture or thread formed of a radiopaque material, such as radiopaque loaded polymeric material or a metallic material. In other embodiments, the radiopaque member 120 is a suture or thread that includes a radiopaque material (such as a radiopaque coating).

For example, in some embodiments, the radiopaque member 120 is a filament formed of or including an object visible with an x-ray device. For example, in some embodiments, the radiopaque member 120 is a metal wire or a wire loaded with a metal or a metal salt. Specifically, in some embodiments, the radiopaque member 120 is a filament formed of or including bismuth, barium, silver, or other heavy metal or heavy metal salt. In some embodiments, the material visible using an x-ray object (i.e., bismuth, barium, silver, or other heavy metal or heavy metal salt) is blended into a polypropylene yarn or thread. In some embodiments, such yarn or thread is integrated into, such as woven into, a knitted fabric or mesh of the substrate 110. In some embodiments, the yarn or thread is used to knit or form the entire implant.

In some embodiments, the radiopaque member 120 is a radiopaque object. For example, in some embodiments, the radiopaque member 120 is a radiopaque object disposed in a carrier. In such embodiments, the carrier can then be disposed on or coupled to the substrate 110. In other embodiments, the radiopaque member 120 is coupled to or applied to the substrate 110 via an adhesive, heat welding, thread through or other known coupling mechanism.

In some embodiments, the radiopaque member 120 is coupled to the substrate 110 in the form or shape of a pattern. For example, in some embodiments, the radiopaque member 120 is coupled to the substrate 110 in the form or shape of a circle or oval. In other embodiments, the radiopaque member 120 is coupled to the substrate 110 in the form or shape of the perimeter of the substrate 110. For example, if the substrate 110 (for example, an outer perimeter of the substrate 110) is an oval shape, the radiopaque member 120 is coupled to the substrate 110 in the shape of an oval which is of a size that is smaller than the oval defined by the outer perimeter of the substrate 110.

In some embodiments, the radiopaque member 120 is coupled to the substrate 110 in the form or shape of a pattern such that a first portion of the pattern is disposed a known distance from another portion of the pattern. In some embodiments, a first portion of the pattern is disposed a first distance from a second portion of the pattern and is disposed a different distance from a third portion of the pattern.

In some embodiments, the radiopaque member 120 includes a first portion that extends along a first axis and a second portion that extends along a second axis. The first axis is different than the second axis. In some embodiment, the first axis is parallel to the second axis. In some embodiments, the first axis is non-parallel to the second axis.

In some embodiments, the radiopaque portion 115 includes multiple radiopaque members. For example, in some embodiments, several radiopaque members are coupled to the substrate to collectively form a pattern (i.e., a radiopaque pattern). For example, in some embodiments, the radiopaque portion 115 includes three radiopaque filaments coupled to the substrate. In some embodiments, the radiopaque members each extend along an axis that is parallel to the others. In some embodiments, the radiopaque members are disposed different but known distances apart from one another. For example, in some embodiments, a first radiopaque member, a second radiopaque member, and a third radiopaque member are coupled to the substrate such that the first radiopaque member is disposed a first known distance from the second radiopaque member and the third radiopaque member is disposed a second known distance from the second radiopaque member. The second distance is different than the first distance. In some embodiments, the second distance is equal, smaller, or greater than the first distance. By measuring the distances through imaging device the physician can then determine the location or deform of the implanted materials.

In use, the implant 100 may be disposed within a body of a patient. For example, as described above, the implant 100 may be placed or disposed within a pelvic region of a patient via an anterior or posterior vaginal incision. In other embodiments, the implant 100 may be placed into another location within a body of a patient through an incision at a different location.

Once the implant 100 is disposed within the body of the patient, an imaging device, such as an x-ray device, may be used to observe the implant 100 within the body of the patient. For example, an x-ray device may be used to detect the location of the radiopaque portion 115 or the radiopaque member 120 within the body of the patient. Specifically, in some embodiments, the pattern of the radiopaque member 120 may be observed or detected using the x-ray device.

The observation or detection of the location of the radiopaque member 120 within the body of the patient may help confirm that the implant 100 is correctly placed within the body of the patient. Additionally, the observation or detection of the pattern of the radiopaque member 120 may allow a physician to determine the status of the implant 100. For example, the observation or detection of the radiopaque member 120 may allow a physician to determine whether the implant 100 has experienced shrinkage, tension, or deformed in any way. Additionally, the observation or detection of the pattern of the radiopaque member 120 may allow a physician to determine an orientation of the implant 100 within the body of the patient. For example the observation or detection of the radiopaque member 120 may allow a physician to determine whether the implant 100 has become folded, twisted, or otherwise misshaped within the body of the patient.

Specifically, in some embodiments, the physician may compare the pattern of the radiopaque member 120 as the pattern existed before the implant 100 was disposed within the body of the patient with the pattern of the radiopaque member 120 as it is observed or detected with an x-ray device after the implant 100 has been placed or disposed within the body of the patient. For example, a physician may compare the distance of a first portion of the pattern from a second portion of the pattern as it existed before the implant 100 was disposed within the body of the patient with the distance of the first portion of the pattern from the second portion of the pattern as it is observed or detected with the x-ray device after the implant 100 has been placed or disposed within the body of the patient.

In some embodiments, the implant 100 may be observed within the body of the patient using an x-ray device just after the implant 100 has been disposed within the body of the patient. For example, the implant 100 may be observed a few minutes or a few hours after the procedure to place the implant 100 within the body has been completed. In other embodiments, the implant 100 may be observed within the body of the patient using an x-ray device long after the procedure to place the implant 100 within the body has been completed. For example, in some embodiments, the implant 100 is observed months, years, or several years, after the implant 100 has been placed within the body of the patient.

In some embodiments, a computer implemented method may be used to determine the status or orientation of the implant 100 within the body of the patient. For example, computer executable code may be stored on a memory device that is computer-readable such as a non-transitory memory device. In some embodiments, the code represents instructions configured to trigger a processor or a computer device or system to perform steps or comparisons to determine the status or orientation of the implant 100 within the body of the patient.

FIG. 2 is a top view of an implant 200 according to an embodiment. The implant 200 includes a substrate 210 and a radiopaque portion 215. The radiopaque portion includes a radiopaque member 220. The implant 200 is configured to be placed with a body of a patient. For example, in some embodiments, the implant 200 is configured to be placed within a body of a patient and configured to provide support to a portion of the body of the patient.

In the illustrated embodiment, the substrate 210 includes arm members 212, 214, 216, and 218. The arm members 212, 214, 216, and 218 are configured to extend to or through bodily tissue to help secure the implant 200 within the body of the patient. In some embodiments, the arm members 212, 214, 216, and 218 include tangs to help secure the implant 200 in place within the body of the patient. In other embodiments, the arm members include barbs, dimples and/or other protrusions configured to engage the bodily tissue of the patient to help retain the implant 200 in place within the body of the patient. In some embodiments, sutures may be used to couple the implant 200 to bodily tissue to help support the implant 200 within the body of the patient.

The substrate 210 can be formed of any material. For example, in some embodiments, the substrate 210 is formed of a biologically compatible material. In some embodiments, the substrate 210 is formed of a mesh material, such as a polypropylene mesh and is configured to allow tissue in-growth to the substrate 210 after implantation within the body of the patient.

The substrate 210 can be monolithically formed or alternatively, the substrate 210 can be formed with multiple different materials and/or can include multiple different components or portions coupled together.

The substrate 210 may be formed of any type of biocompatible material, such as a biocompatible synthetic material or a natural, biologic material. In some embodiments, the substrate 210 is formed of a biocompatible synthetic material. In some embodiments, the substrate 210 can be formed with a combination of materials including synthetic and biological materials. For example, in some embodiments, a body portion of the substrate 210 may be formed of a natural or biologic material and the arm members may be formed of a synthetic material. In other embodiments, the substrate 210 is formed with a natural or biological material.

The radiopaque member 220 is coupled to the substrate 210. In some embodiments, the radiopaque member 220 is a filament such as a suture or a thread. For example, in some embodiments, the radiopaque member 220 is a suture or thread formed of a radiopaque material. In other embodiments, the radiopaque member 220 is a suture or thread that includes a radiopaque material (such as a radiopaque coating).

In the illustrated embodiment, the radiopaque member 220 is coupled to the substrate 210 in the form or shape of a pattern. The pattern includes several portions or locations. Specifically, a first portion A of the pattern is disposed a known distance from a second portion B of the pattern. The first portion A of the pattern is disposed a second distance from a third portion C of the pattern. Additionally, the second portion B of the pattern is disposed a third distance from the third portion C of the pattern. In the illustrated embodiment, the first distance is greater than the second distance and the second distance is greater than the third distance.

The radiopaque member 220 includes a first portion 220 that extends along a first axis A1. The radiopaque member 220 also includes a second portion 224 that extends along a second axis A2. The first axis A1 is different than the second axis A2. The first axis A1 is angled with respect to the second axis A2 (i.e., the first axis A1 is not parallel to the second axis A2).

In use, the implant 200 may be disposed within a body of a patient using a known method. For example, as described above, the implant 200 may be placed or disposed within a pelvic region of a patient via an anterior or posterior vaginal incision. In other embodiments, the implant 200 may be placed into another location within a body of a patient through an incision at a different location.

Once the implant 200 is disposed within the body of the patient, an x-ray device may be used to observe the implant 200 within the body of the patient. For example, an x-ray device may be used to detect the location of the radiopaque member 220 within the body of the patient. Specifically, in some embodiments, the pattern of the radiopaque member 220 may be observed or detected using the x-ray device.

The physician or other user may then compare the pattern of the radiopaque member 220 as the pattern existed before the implant 200 was disposed within the body of the patient with the pattern of the radiopaque member 220 as it is observed or detected with an x-ray device after the implant 200 has been placed or disposed within the body of the patient. A physician or other user may also compare the first distance, the second distance, and the third distance as they existed before the implant 200 was disposed within the body of the patient with the first distance, the second distance, and the third distance as they are observed or detected with the x-ray device after the implant 200 has been placed or disposed within the body of the patient. Accordingly, the physician or other user may be able to determine whether the implant 200 has moved from its original or desired position within the body of the patient. Additionally, the physician or other user may be able to determine the orientation of the implant 200 (for example, whether the implant 200 has been folded on top of itself) and whether any portion of the implant 200 has experienced any shrinkage.

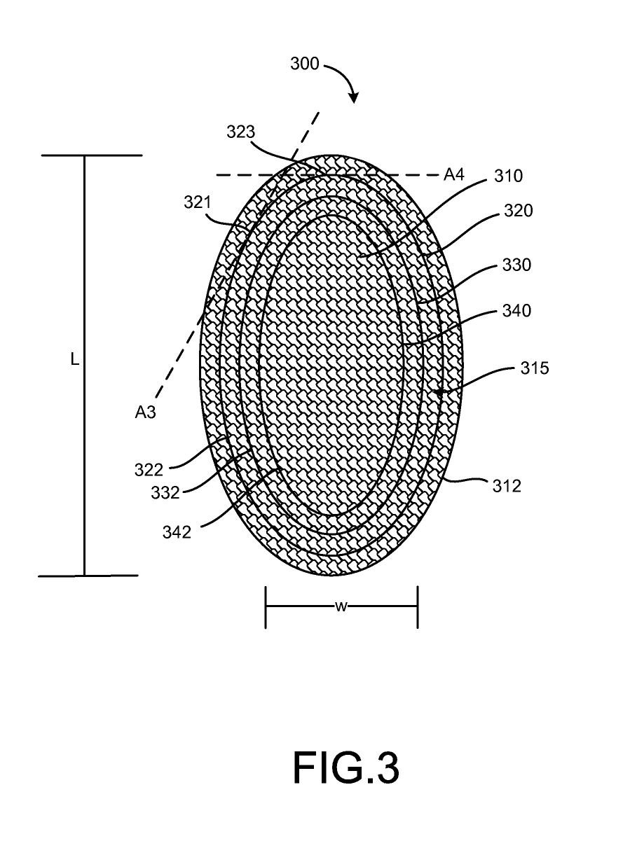

FIG. 3 is a top view of an implant 300 according to an embodiment. The implant 300 includes a substrate 310 and a radiopaque portion 315. The radiopaque portion 315 includes a first radiopaque member 320, a second radiopaque member 330, and a third radiopaque member 340. The implant 300 is configured to be placed with a body of a patient. For example, in some embodiments, the implant 300 is configured to be placed within a body of a patient and configured to provide support to a portion of the body of the patient. For example, the implant 300 may be placed within a pelvic region of a patient and may provide support to a bladder, a uterus or another portion of the body of the patient.

In the illustrated embodiment, the substrate 310 is formed of a mesh material, such as a polypropylene mesh and is configured to allow tissue in-growth to the substrate 310 after implantation within the body of the patient.

The second radiopaque member 330 and the third radiopaque member 340 are structurally and functionally similar to the first radiopaque member 320. Accordingly, only the first radiopaque member 420 will be discussed in detail. The first radiopaque member 320 is coupled to the substrate 310. In the illustrated embodiment, the radiopaque member 320 is a filament such as a suture or a thread. For example, in some embodiments, the radiopaque member 320 is a suture or thread formed of a radiopaque material. In other embodiments, the radiopaque member 320 is a suture or thread that includes a radiopaque material (such as a radiopaque coating).

The first radiopaque member 320 is coupled to the substrate 210. For example, in some embodiments, the first radiopaque member 320 is woven to the mesh of the substrate 310. In other embodiments, another mechanism is used to couple the first radiopaque member 320 to the substrate 310. For example, the first radiopaque member 320 maybe coupled to the substrate 310 via an adhesive or a welding process, such as a heat welding process.

The substrate 310 includes a perimeter 312 that defines a shape. In the illustrated embodiment, the perimeter 312 defines an oval shape of a size. Specifically, in the illustrated embodiment, the perimeter 312 defines an oval shape that has a length of L and a width of W. The oval shape can have any length L and width W. For example, in some embodiments, the oval shape may be 2 cm by 7 cm, 4 cm by 7 cm, 6 cm by 10 cm, 6 cm by 12 cm, 8 cm by 12 cm, or 10 cm by 15 cm. In other embodiments, the perimeter of the substrate defines a different shape.

In the illustrated embodiment, the first radiopaque member 320 includes a first portion 321 that extends along a first axis A3. The first radiopaque member 320 also includes a second portion 323 that extends along a second axis A4. The first axis A3 is different than the second axis A4 and is angled with respect to the second axis A4.

In the illustrated embodiment, the first radiopaque member 320, the second radiopaque member 330, and the third radiopaque member 340 are coupled to the substrate 310 and collectively form a pattern. Specifically, in the illustrated embodiment, the first radiopaque member 320, the second radiopaque member 330, and the third radiopaque member 340 are each in the form or shape of ovals. In the illustrated embodiment, the radiopaque members 320, 330, and 340 are concentric ovals.

In the illustrated embodiment, the first radiopaque member 320 is coupled to the substrate 310 and forms a shape that is the same as or similar to the shape defined by the perimeter 312 of the substrate 310. Specifically, the first radiopaque member 320 is coupled to the substrate 310 and forms an oval shape that has a length that is smaller than L (the length of the oval defined by the perimeter 312 of the substrate 310) and a width that is smaller than W (the width of the oval defined by the perimeter 312 of the substrate 310).

In the illustrated embodiment, the second radiopaque member 330 is coupled to the substrate 310 and forms a shape that is the same as or similar to the shape defined by the perimeter 312 of the substrate 310. Specifically, the second radiopaque member 330 is coupled to the substrate 310 and forms an oval shape that has a length that is smaller than the length of the oval of the first radiopaque member 320 and a width that is smaller than the width of the oval of the first radiopaque member 320.

In the illustrated embodiment, the third radiopaque member 340 is coupled to the substrate 310 and forms a shape that is the same as or similar to the shape defined by the perimeter 312 of the substrate 310 and of a size that is smaller than the size of the shape defined by the perimeter 312 of the substrate 310. Specifically, the third radiopaque member 340 is coupled to the substrate 310 and forms an oval shape that has a length that is smaller than the length of the oval of the second radiopaque member 330 and a width that is smaller than the width of the oval of the second radiopaque member 330.

The pattern formed by the radiopaque members 320, 330, and 340 includes several portions or locations. Specifically, a portion 322 of the first radiopaque member 320 is disposed a known distance from a portion 332 of the second radiopaque member 330. The portion 322 of the first radiopaque member 320 is disposed a second distance from a portion 342 of the third radiopaque member 330. Additionally, the portion 332 of the second radiopaque member 330 is disposed a third distance from the portion 342 of the third radiopaque member 340.

In some embodiments, the first distance is greater than the second distance and the second distance is greater than the third distance. In other embodiments, the first distance, the second distance, and the third distance are all substantially the same.

In some embodiments, the particular pattern of the radiopaque member facilitates or allows a physician to cut or trim the implant to a size suitable for a particular patient. For example, in the illustrated embodiment, the implant 300 may be cut or trimmed between the perimeter 312 of the substrate 310 and the first radiopaque member 320. Similarly, in the illustrated embodiment, the implant 300 may be cut or trimmed between the first radiopaque member 320 and the second radiopaque member 330. Accordingly, the trimmed implant 300 (that includes the substrate 310, the second radiopaque member 330, and the third radiopaque member 340) may be placed within a body of a patient.

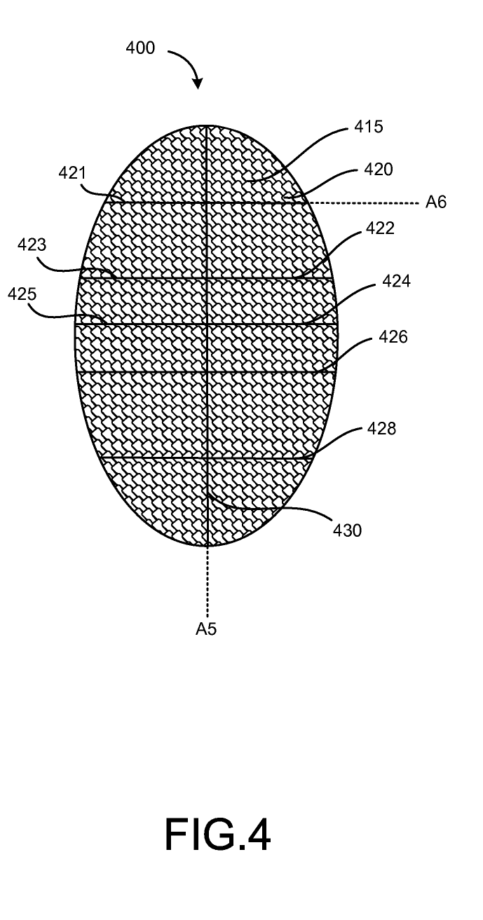

FIG. 4 is a top view of an implant 400 according to an embodiment. The implant 400 includes a substrate 410 and a radiopaque portion 415. The radiopaque portion 415 includes radiopaque members 420, 422, 424, 426, 428, and 430. The implant 400 is configured to be placed with a body of a patient. For example, in some embodiments, the implant 400 is configured to be placed within a body of a patient and configured to provide support to a portion of the body of the patient. For example, the implant 400 may be placed within a pelvic region of a patient and may provide support to a bladder, a uterus or another portion of the body of the patient.

In the illustrated embodiment, the substrate 410 is formed of a mesh material, such as a polypropylene mesh and is configured to allow tissue in-growth to the substrate 410 after implantation within the body of the patient.

The radiopaque members 422, 424, 426, 428, and 430 are structurally and functionally similar to the radiopaque member 420. Accordingly, only radiopaque member 420 will be discussed in detail. The radiopaque member 420 is coupled to the substrate 410. In the illustrated embodiment, the radiopaque member 420 is a filament such as a suture or a thread. For example, in some embodiments, the radiopaque member 420 is a suture or thread formed of a radiopaque material. In other embodiments, the radiopaque member 420 is a suture or thread that includes a radiopaque material (such as a radiopaque coating).

The radiopaque member 420 is coupled to the substrate 410. For example, in some embodiments, the radiopaque member 420 is woven to the mesh of the substrate 410. In other embodiments, another mechanism is used to couple the radiopaque member 420 to the substrate 410. For example, the radiopaque member 420 maybe coupled to the substrate 410 via an adhesive or a welding process, such as a heat welding process.

In the illustrated embodiment, the radiopaque members 420, 422, 424, 426 428, and 430 collectively form a pattern. Radiopaque member 430 extends along the center of the implant 400 and along a longitudinal axis A5 of the implant 400. Radiopaque member 420 extends along axis A6 which is angled with respect to axis A5. Specifically, axis A6 is perpendicular to axis A5. Additionally, radiopaque members 422, 424, 426, and 428 extend perpendicular to radiopaque member 430. Specifically, the radiopaque members 420, 422, 424, 426, and 428 all cross radiopaque member 430 at a different location on the implant 400.

The radiopaque members 420, 422, 424, 426, and 428 are disposed apart from one another at different distances. For example, in the illustrated embodiment, radiopaque member 420 is disposed a first distance from radiopaque member 422 and radiopaque member 422 is disposed a second distance different than the first distance from radiopaque member 424. Specifically, a portion 421 of radiopaque member 420 is disposed a first distance from a portion 423 of radiopaque member 422. Portion 423 of radiopaque member 422 is disposed a second distance from portion 425 of radiopaque member 424. The second distance is less than the first distance. The observation or detection of if the radiopaque member 430 is straight may allow a physician to determine if the mesh is flat as it supposes to be within the body of the patient.

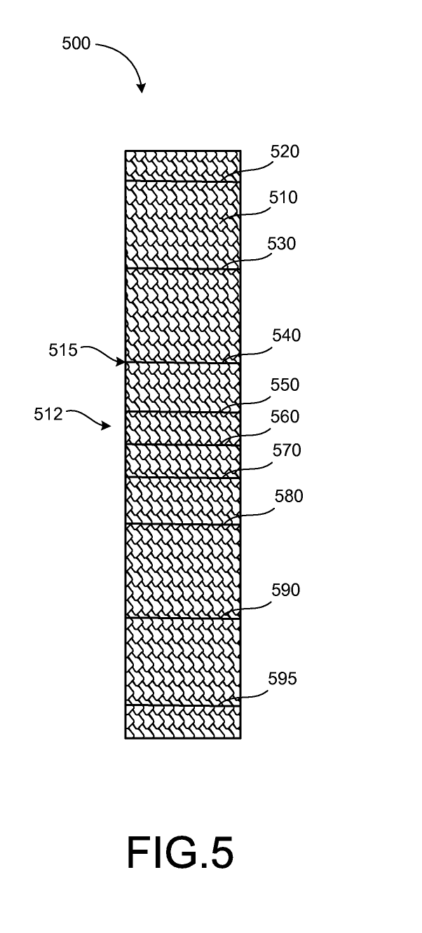

FIG. 5 is a top view of an implant 500 according to an embodiment. The implant 500 includes a substrate 510 and a radiopaque portion 515. The radiopaque portion includes radiopaque members 520, 530, 540, 550, 560, 570, 580, 590, and 595. The implant 500 is configured to be placed with a body of a patient. For example, in some embodiments, the implant 500 is configured to be placed within a body of a patient and configured to provide support to a portion of the body of the patient. For example, the implant 500 may be placed within a pelvic region of a patient and may provide support to a bladder, a uterus or another portion of the body of the patient.

In the illustrated embodiment, the substrate 510 is formed of a mesh material, such as a polypropylene mesh and is configured to allow tissue in-growth to the substrate 510 after implantation within the body of the patient. In the illustrated embodiment, the substrate 510 is rectangular and extends along a longitudinal axis.

The radiopaque members 530, 540, 550, 560, 570, 580, 590, and 595 are structurally and functionally similar to the radiopaque member 520. Accordingly, only radiopaque member 520 will be discussed in detail. The radiopaque member 520 is coupled to the substrate 510. In the illustrated embodiment, the radiopaque member 520 is a filament such as a suture or a thread. For example, in some embodiments, the radiopaque member 520 is a suture or thread formed of a radiopaque material. In other embodiments, the radiopaque member 520 is a suture or thread that includes a radiopaque material (such as a radiopaque coating).

The radiopaque member 520 is coupled to the substrate 510. For example, in some embodiments, the radiopaque member 520 is woven to the mesh of the substrate 510. In other embodiments, another mechanism is used to couple the radiopaque member 520 to the substrate 510. For example, the radiopaque member 520 maybe coupled to the substrate 510 via an adhesive or a welding process, such as a heat welding process.

In the illustrated embodiment, the radiopaque members 520, 530, 540, 550, 560, 570, 580, 590, and 595 collectively form a pattern. Specifically, the radiopaque members 520, 530, 540, 550, 560, 570, 580, 590, and 595 extend perpendicularly to a longitudinal axis defined by the substrate 510. The radiopaque members 520, 530, 540, 550, 560, 570, 580, 590, and 595 are all disposed in parallel directions and are disposed different distances apart. For example, radiopaque member 520 is disposed a first distance from radiopaque member 530 and radiopaque member 530 is disposed a second distance (different than the first distance) from radiopaque member 540. In the illustrated embodiment, the radiopaque members are disposed closer together near a middle portion 512 of the substrate 510.

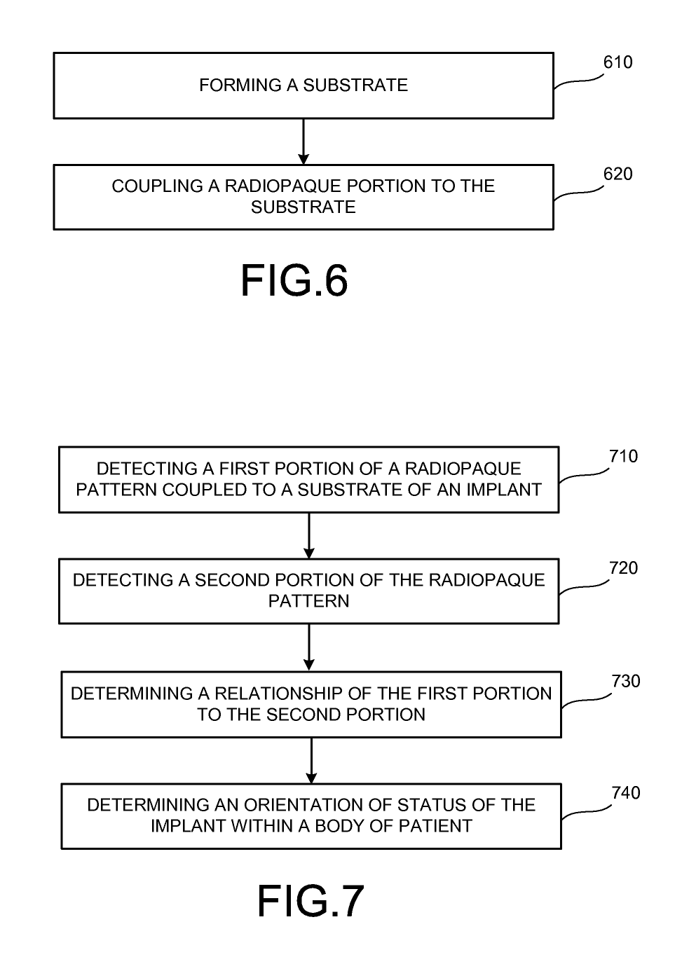

FIG. 6 is a flow chart of a method 600 for forming a bodily implant. The method includes at step 610 forming a substrate. The substrate can be formed by knitting a synthetic mesh and cutting to the mesh to a desired shape. In other embodiments, the substrate can be formed by cutting another natural or synthetic material to a desired shape and size.

At step 620, a radiopaque portion is coupled to the substrate such that the radiopaque portion has a first portion of the radiopaque portion extends along a first axis and a second portion of the radiopaque portion extends along a second axis different than the first axis. In some embodiments, the radiopaque portion is woven to the substrate. In other embodiments, another method of coupling the radiopaque portion to the substrate is used. In some embodiments, the radiopaque portion includes a plurality of radiopaque fibers.

In some embodiments, the coupling the radiopaque portion to the substrate includes coupling a first radiopaque member, a second radiopaque member, and a third radiopaque member to the substrate such that the first radiopaque member is a first distance from the second radiopaque member and the second radiopaque member is disposed a second distance from the third radiopaque member. In some embodiments, the second distance is greater than the first distance.

FIG. 7 is a flow chart of a method 700 of determining an orientation of an implant within a body of a patient. In some embodiments, the method includes at step 710 detecting a location of a first portion of a radiopaque pattern coupled a substrate of an implant disposed within a body of a patient. In some embodiments, this detecting may be done with an imaging device such as an x-ray machine or device.

At step 720, the method includes detecting a location of a second portion of the radiopaque pattern. In some embodiments, the first portion and the second portion of the radiopaque pattern are portions of a single radiopaque member that is coupled to the substrate of the implant. In other embodiments, the first portion of the radiopaque pattern is a portion of a first radiopaque member coupled to the substrate of the implant and the second portion of the radiopaque pattern is a portion of a second radiopaque member (different than the first radiopaque member) that is coupled to the substrate of the implant.

At step 730, the method includes determining a relationship of the first portion of the radiopaque pattern to the second portion of the radiopaque pattern while the implant is disposed within the body of the patient. In some embodiments, this determining includes a comparison of the location of the first portion of the radiopaque pattern to the location of the second portion of the radiopaque pattern. In some embodiments, this determining includes determining the relative location of the first portion of the radiopaque pattern to the second portion of the radiopaque pattern (i.e., the location of the first portion of the radiopaque pattern with respect to the second portion of the radiopaque pattern).

At step 740, in some embodiments, the method includes determining an orientation of the implant within the body of the patient based on a comparison of a relationship of the first portion of the radiopaque pattern to the second portion of the radiopaque pattern before the implant was disposed within the body of the patient to the relationship of the first portion of the radiopaque radiopaque member to the second radiopaque member after the implant was disposed within the body of the patient. For example, in some embodiments, the relationship of the first portion of the radiopaque pattern to the second portion of the radiopaque pattern is known at the time the implant is disposed within the body of the patient. Once the relationship of the first portion of the radiopaque pattern to the second portion of the radiopaque pattern while the implant is disposed within the body of the patient is determined, this relationship can be compared to the relationship of the portions before the implant was disposed within the body of the patient. Any differences in the relationships can identify the orientation (or status, or location) of the implant within the body of the patient.

In some embodiments, the disclosed invention (in whole or in part) is accomplished using a computer (a general purpose computer or a computer dedicated for the performance of the disclosed invention such as an embedded appliance) or a network of computers. In some embodiments, the computer includes at least one processor component and at least one memory component. In some embodiments, the memory component may be incorporated into the processor component. The memory may be configured to store code which, when executed, causes the computer to perform the steps of the invention. For example, the computer may function to do data processing, detecting, or determining, or any combination thereof. In other embodiments, the computer may function to perform other steps or processes of the invention.

In other embodiments of the invention, code is stored on a computer readable medium which when executed by a computer causes the computer to perform steps or processes of the disclosed invention. For example, the code may be stored on any medium readable by a computer, such as a DVD, CD, or other memory device (e.g., flash memory, non-transitory memory device).

In some embodiments, the computer also functions to output to a user the results of a process or processes performed by the computer. For example, the computer (or code) may be configured to trigger display of the results of the process or processes to a user via a computer monitor, computer print out, or other computer output.

In one embodiment, an implant includes a substrate and a radiopaque portion coupled to the substrate. The radiopaque portion has a first portion extending along a first axis and a second portion extending along a second axis different than the first axis.

In some embodiment, the substrate is a mesh material and the radiopaque portion includes a radiopaque filament that is woven to the mesh material. In one embodiment, the radiopaque portion includes a first radiopaque filament and a second radiopaque filament. The first radiopaque filament is coupled to the substrate such that it extends along the first axis. The second radiopaque filament is coupled to the substrate such that it extends along the second axis.

In some embodiments, the radiopaque portion includes a third portion that extends along a third axis different than the first axis and the second axis.

In some embodiments, the radiopaque portion forms a circular shape. In some embodiments, the substrate has a perimeter that defines a shape having a first size. The radiopaque portion forms a shape having a second size. The shape formed by the radiopaque portion is substantially the same as the shape defined by the perimeter of the substrate. The first size being greater than the second size.

In some embodiments, the radiopaque portion includes a first radiopaque filament, a second radiopaque filament, and a third radiopaque filament. The first radiopaque filament is coupled to the substrate and extends along the first axis. The second radiopaque filament is coupled to the substrate and extends along the second axis. The third filament is coupled to the substrate and extends along a third axis different than the first axis and the second axis.

In some embodiments, an implant includes a substrate, a first radiopaque filament, a second radiopaque filament, and a third radiopaque filament. The first radiopaque filament is coupled to the substrate. The second radiopaque filament is coupled to the substrate and is disposed a first distance from the first radiopaque filament. The third radiopaque filament is coupled to the substrate and is disposed a second distance from the second radiopaque filament. The first distance is greater than the second distance.

In some embodiments, the substrate has a perimeter that defines a shape that has a first size. The first radiopaque filament forms a shape having a second size. The second radiopaque filament forms a shape having a third size. The shaped formed by the first radiopaque filament is substantially the same as the shape defined by the perimeter of the substrate. The shaped formed by the first radiopaque filament is substantially the same as the shaped formed by the second radiopaque filament. The first size is greater than the second size. The second size is greater than the third size.

In some embodiments, the first radiopaque filament extends along a first axis. The second radiopaque filament extends along a second axis. The first axis is substantially parallel to the second axis.

In some embodiments, the first radiopaque filament extends along a first axis. The second radiopaque filament extends along a second axis. The first axis is substantially parallel to the second axis. The third radiopaque filament extends along a third axis. The third axis is parallel to the first axis and parallel to the second axis.

In one embodiment, a method of forming a bodily implant includes (1) forming a substrate, and (2) coupling a radiopaque portion to the substrate such that the radiopaque portion has a first portion extending along a first axis and a second portion extending along a second axis different than the first axis.

In some embodiments, the substrate is a mesh substrate and the coupling a radiopaque portion to the substrate includes weaving the radiopaque portion into the mesh. In some embodiments, the coupling a radiopaque portion to the substrate includes coupling a first radiopaque member to the substrate and coupling a second radiopaque member to the substrate. In some embodiments, the coupling a radiopaque portion to the substrate includes coupling a first radiopaque filament to the substrate and coupling a second radiopaque filament to the substrate.

In some embodiments, the forming a substrate includes forming the substrate such that the substrate has a perimeter defining a shape and the coupling the radiopaque portion to the substrate includes coupling the radiopaque portion to the substrate such that the radiopaque portion forms a shape substantially the same as the shape defined by the perimeter of the substrate.

In one embodiment, a method includes (1) detecting a location of a first portion of a radiopaque pattern coupled a substrate of an implant disposed within a body of a patient, (2) detecting a location of a second portion of the radiopaque pattern, and (3) determining a relationship of the first portion of the radiopaque pattern to the second portion of the radiopaque pattern while the implant is disposed within the body of the patient.

In some embodiments, the method also includes determining an orientation or status of the implant within the body of the patient based on a comparison of a relationship of the first portion of the radiopaque pattern to the second portion of the radiopaque pattern before the implant was disposed within the body of the patient to the relationship of the first portion of the radiopaque member to the second radiopaque member after the implant was disposed within the body of the patient.

In some embodiments, the detecting a location of a first portion of the radiopaque pattern coupled to the substrate of an implant disposed within the body of the patient includes using an x-ray device to determine the location.

In some embodiments, the method includes exposing the body of the patient to radiation of an x-ray machine.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implant, comprising:
a substrate;
a radiopaque portion coupled to the substrate, the radiopaque portion having a first portion extending along a first axis and a second portion extending along a second axis different than the first axis.

2. The implant of claim 1, wherein the substrate is a mesh material, the radiopaque portion includes a radiopaque filament that is woven to the mesh material.

3. The implant of claim 1, wherein the radiopaque portion including a first radiopaque filament and a second radiopaque filament, the first radiopaque filament is coupled to the substrate such that it extends along the first axis, the second radiopaque filament is coupled to the substrate such that it extends along the second axis.

4. The implant of claim 1, wherein the radiopaque portion includes a third portion that extends along a third axis different than the first axis and the second axis.

5. The implant of claim 1, wherein the radiopaque portion forms a circular shape.

6. The implant of claim 1, wherein the substrate has a perimeter defining a shape having a first size, the radiopaque portion forming a shape having a second size, the shape formed by the radiopaque portion being substantially the same as the shape defined by the perimeter of the substrate, the first size being greater than the second size.

7. The implant of claim 1, wherein the radiopaque portion includes a first radiopaque filament, a second radiopaque filament, and a third radiopaque filament, the first radiopaque filament being coupled to the substrate and extending along the first axis, the second radiopaque filament being coupled to the substrate and extending along the second axis, the third filament being coupled to the substrate and extending along a third axis different than the first axis and the second axis.

8. An implant, comprising:
a substrate;
a first radiopaque filament coupled to the substrate;
a second radiopaque filament coupled to the substrate and being disposed a first distance from the first radiopaque filament; and
a third radiopaque filament coupled to the substrate and being disposed a second distance from the second radiopaque filament, the first distance being greater than the second distance.

9. The implant of claim 8, wherein the substrate has a perimeter defining a shape having a first size, the first radiopaque filament forming a shape having a second size, the second radiopaque filament forming a shape having a third size, the shaped formed by the first radiopaque filament being substantially the same as the shape defined by the perimeter of the substrate, the shaped formed by the first radiopaque filament being substantially the same as the shaped formed by the second radiopaque filament, the first size being greater than the second size, the second size being greater than the third size.

10. The implant of claim 8, wherein the first radiopaque filament extends along a first axis, the second radiopaque filament extends along a second axis, the first axis being substantially parallel to the second axis.

11. The implant of claim 8, wherein the first radiopaque filament extends along a first axis, the second radiopaque filament extends along a second axis, the first axis being substantially parallel to the second axis, the third radiopaque filament extends along a third axis, the third axis being parallel to the first axis and parallel to the second axis.

12. A method of forming a bodily implant, comprising:
forming a substrate;
coupling a radiopaque portion to the substrate such that the radiopaque portion has a first portion extending along a first axis and a second portion extending along a second axis different than the first axis.

13. The method of claim 12, wherein the substrate is a mesh substrate, the coupling a coupling a radiopaque portion to the substrate includes weaving the radiopaque portion into the mesh.

14. The method of claim 12, wherein the coupling a radiopaque portion to the substrate includes coupling a first radiopaque member to the substrate and coupling a second radiopaque member to the substrate.

15. The method of claim 12, wherein coupling a radiopaque portion to the substrate includes coupling a first radiopaque filament to the substrate and coupling a second radiopaque filament to the substrate

16. The method of claim 12, wherein the forming a substrate includes forming the substrate such that the substrate has a perimeter defining a shape, the coupling the radiopaque portion to the substrate includes coupling the radiopaque portion to the substrate such that the radiopaque portion forms a shape substantially the same as the shape defined by the perimeter of the substrate.

17. A method, comprising:
detecting a location of a first portion of a radiopaque pattern coupled a substrate of an implant disposed within a body of a patient;
detecting a location of a second portion of the radiopaque pattern;
determining a relationship of the first portion of the radiopaque pattern to the second portion of the radiopaque pattern while the implant is disposed within the body of the patient.

18. The method of claim 17, further comprising:
determining an orientation or status of the implant within the body of the patient based on a comparison of a relationship of the first portion of the radiopaque pattern to the second portion of the radiopaque pattern before the implant was disposed within the body of the patient to the relationship of the first portion of the radiopaque radiopaque member to the second radiopaque member after the implant was disposed within the body of the patient.

19. The method of claim 17, wherein the detecting a location of a first portion of the radiopaque pattern coupled to the substrate of an implant disposed within the body of the patient includes using an x-ray device to determine the location.

20. The method of claim 17, further comprising:
exposing the body of the patient to radiation of an x-ray machine.
